(19) United States
(12) Patent Application Publication
(10) Pub. No.: US 2006/0155004 A1
(43) Pub. Date: Jul. 13, 2006

(54) Resin mortar composition for construction and floor operating method using the same

(76) Inventor: Sang-Woon Kwak, Seoul (KR)

Correspondence Address: CANTOR COLBURN, LLP, 55 GRIFFIN ROAD SOUTH, BLOOMFIELD, CT, 06002, US

(21) Appl. No.: 10/526,454

(22) PCT Filed: Sep. 4, 2003
(86) PCT No.: PCT/KR03/01812

§ 371 Date: Oct. 24, 2005

(30) Foreign Application Priority Data
Sep. 5, 2002 (KR) 10-2002-0053534

(51) Int. Cl. A61K 6/08

(52) U.S. Cl. 523/117

(57) ABSTRACT

The present invention relates to a resin mortar composition, which has excellent flowing property, storage property, excellent self-leveling property when constructed, and excellent abrasion resistance, surface scratch resistance, crack resistance and durability after construction, which can be easily constructed and easily control curing time, and is economical. According to the present invention, there are provided a resin mortar composition for construction, which comprises, on the basis of solid content, a) 100 parts by weight of room temperature curable organic liquid phase resin; b) 10 to 200 parts by weight of glass bead, c) 10 to 400 parts by weight of glass powder, and d) 0.1 to 50 parts by weight of glass fiber, and a method for constructing a floor that has beautiful surface and does not show crack using the same.

### BACKGROUND OF THE INVENTION

(a) Field of the Invention

The present invention relates to a resin mortar composition that has excellent flowing property, storage property, self-leveling property when constructed, and excellent abrasion resistance, surface scratch resistance, crack resistance and durability after constructed, can be easily constructed and easily control curing time, and is economical, and a method for constructing a floor that has beautiful surface and does not show crack using the same.

(b) Description of the Related Art

Conventional resin mortar comprises a liquid phase resin and a filler such as quartz sand, and it is constructed on a floor by mixing it with a curing agent in an appropriate mixing ratio while agitating, mixing quarts sand therewith, coating the mixture on a construction surface with a specific thickness, decorating it using a decoration apparatus, and repeatedly constructing and curing additional resin until a uniform coating forms, in a construction field. Generally, a resin is mixed with fillers such as quartz sand, the quartz sand absorbs the resin hence a viscosity largely increases making it difficult to be uniformly dispersed, and even if uniformly dispersed, phase separation of the resin and quartz sand easily occurs. Therefore, a resin mortar should be mixed in a construction field. Since such a mixing in a construction field should be conducted manually with small amount at a time, treated amount is limited and a lot of hands and equipment are required in order to construct a large area, making it inefficient and increasing construction cost.

In addition, in the case of a resin mortar containing quartz sand as fillers, since the quartz sand absorbs the resin, the absolute amount of the filler functioning for bonding is insufficient and resin is prevented from remaining on the surface of quartz sand, which decreases strength and causes damages.

In addition, in order to facilitate construction, the amount of filler for resin should be limited, and if the amount of filer increases, operability is not good and construction is difficult. And, in case a filler covered with a small amount of resin is contained, it is directly exposed as time passes by, making it exposed to various impacts. In addition, after construction, if quartz sand used as a filler is exposed or an upper coating film is damaged by various causes during usage, the surface of quartz sand is contaminated to make the surface appearance inferior, and since cleaning of quartz sand is impossible because of its property, contaminants are absorbed into the pores of quartz sand or inside of it, which causes offensive odor and bacterial growth, and if water or oil is introduced for a long period, surface separation of a resin and a filler may be caused.

Meanwhile, a lining operation, which is a kind of conventional self-leveling for building, uses talc or calcium carbonate, etc. as a filler hence it has good flowing property. However, since it requires comparatively expensive resin, it is uneconomical. And, after construction, hardness, compression strength and adhesion strength are low and thus adhesion, durability, scratch resistance, etc. are inferior. In addition, inhalation property for water, oil, etc. and contamination resistance are identical to general resin mortar, and if water or oil remains on its surface, it becomes very slippery to cause safety accident.

### SUMMARY OF THE INVENTION

It is an object of the present invention to provide a resin mortar composition having excellent flowing property, storage property and self-leveling property when constructed.

It is another object of the present invention to provide a resin mortar composition having excellent abrasion resistance, surface scratch resistance, crack resistance and durability after constructed.

It is another object of the present invention to provide a resin mortar composition that can be easily constructed, can easily control curing time and is economical.

It is another object of the present invention to provide a method for constructing a floor that has beautiful surface and does not show crack using the resin mortar composition.

In order to achieve these objects, the present invention provides a resin mortar composition for construction, which comprises:
on the basis of solid contents,
a) 100 parts by weight of a room temperature curable organic liquid phase resin;
b) 100 to 200 parts by weight of glass beads;
c) 10 to 400 parts by weight of glass powder; and
d) 0.1 to 50 parts by weight of glass fiber.

The present invention also provides a method for constructing a resin mortar, which comprises the steps of:

a) coating a resin mortar comprising:
i) 100 parts by weight of a room temperature curable organic liquid phase resin;
ii) 10 to 200 parts by weight of glass beads;
iii) 10 to 400 parts by weight of glass powder; and
iv) 1 to 50 parts by weight of glass fiber on a floor;

b) spraying glass beads on the floor on which the resin mortar is coated to remove bubbles produced in the coated resin mortar; and

c) curing the resin mortar floor from which bubbles are removed.

### BRIEF DESCRIPTION OF THE DRAWINGS

FIG. 1 is a cross-sectional view showing one example of resin mortar of the present invention constructed on a bottom surface of cement concrete.

FIG. 2 is a photograph showing abrasion resistance results of cured resin mortar of the present invention.

Reference numeral 1 denotes resin, 2 filler, 2a glass beads, 2b glass powder, 2c glass fiber, 3 a cement bottom surface, 10 a sample before abrasion resistance test, and 20 a sample after abrasion resistance test.

### DETAILED DESCRIPTION OF THE PREFERRED EMBODIMENTS

The present inventors have discovered that if glass beads are contained in a resin mortar as fillers, even if a large amount is contained, a volume filling property and compatibility are superior and flowing property of resin mortar significantly increases, and completed the present invention.

The present invention blends a room temperature curable organic liquid phase resin such as epoxy based, acryl based, urethane based, alkyd based, polyester based, or polyvinylchloride based resin, etc. with glass beads, glass powder and milled glass fiber as fillers while agitating, thereby providing a resin mortar composition that has superior flowing property, storage property and self-leveling property when constructed, and superior abrasion resistance, surface scratch resistance, crack resistance, weather resistance and durability after constructed, and that can be easily constructed, can easily control curing time, and is economical. Such a resin mortar does not generate voids because of complicated bonding of small glass powder or milled glass fiber that fill voids between glass beads of various sizes, has superior impact resistance because of buffering effects due to pressure dispersion of glass beads against external impact, and has superior flowing property because viscosity does not increase hence remarkably improving construction operability.

As the room temperature curable organic liquid phase resin used in the resin mortar of the present invention, conventionally used epoxy based, acryl based, urethane based, alkyd based, polyester based, or polyvinylchloride based resins can be used. As the epoxy based resin, a solvent or non-solvent type diglycidyl type or triglycidyl type epoxy resin that has molecular weight of 350 to 3,000 MW is preferable. As the acryl based resin, a solvent type acryl urethane that has methacrylic acid derivatives as main ingredients, an aqueous acrylhydrosol, an emulsion non-solvent type acryl silane, or a UV curable acryl is preferable. As the alkyd based resin, paint shaped alkyd resin that is modified with polyacidic base and polyhydric alcohol ester compounds is preferable, and alkyd resins modified with rosin, phenol, epoxy, vinyl styrene monomer, isocyanate or silicon can be used. As the polyvinyl chloride based resin, a PVC plastic sol liquid phase resin is preferable.

These resins function as a binder of the resin mortar, and confer acid resistance and alkali resistance. And, if necessary, they can be cured by adding a curing agent, and a cure-promoting agent can be used in order to control curing speed. The curing agent and cure-promoting agent are selected according to the kinds and amounts of the resins, and the amount thereof can be determined considering the use of the constructed floor and construction conditions.

If the content of the room temperature curable liquid phase resin is too low, its function as a binder is insufficient, and if the content is too high, the content of a filler decreases and thus hardness, strength and other physical properties as a floor are inferior. Therefore, the resin and the filler should be blended with the above-mentioned ratio.

Since the glass beads used in the present invention do not have resin absorbing property differently from silica conventionally used as a filler, even if a large amount is used, mixing and dispersion is good and volume filling effect is superior. Particularly, it gives high flowing property to a resin mortar due to the spherical effects of glass beads, it also gives self-leveling property, and it provides superior storage property such that a resin composition comprising a resin and fillers is well mixed by a simple agitation even after stored for a long period. In addition, since the glass beads have higher hardness than silica, they increase surface hardness after a resin mortar is cured to give abrasion resistance, and they provide a surface property of scratch resistance and surface contamination preventing effect. And, when an impact is applied to a cured resin mortar, the glass beads disperse pressure to give high impact resistance because they have spherical or similar shapes. And, the glass beads make an expensive resin mortar economical due to high volume filling properties.

In addition, the glass beads give flame-retarding property to a resin mortar because they are inflammable, and they inhibit generation of static electricity to prevent surface contamination of a constructed resin mortar. And, since the glass beads are made of glasses, they give transparent or white color to a resin mortar, and they give various colors or various patterns such as granite pattern together with other pigments or color chips. And, they can use a large amount of sunlight or irradiated light from a separate lighting to cause diffused reflection thereby decreasing gloss produced from a resin.

As the glass beads used in the present invention, those of spherical, oval or similar shapes can be used, and those having various size distribution or those having a specific size can be used. However, the size of the glass bead is preferably selected according to the use of constructed floor and constructed thickness. Preferably, the sized of glass beads are 200 meshes to 3 mm. If particles of less than 200 meshes are used, volume filling property and impact resistance may deteriorate. If particles of more than 3 mm are used, dispersion may be deteriorated or a resin mortar may be protruded because a resin mortar is coated with a thickness of approximately 0.3 to 10 mm. And, appropriate color can be given to a resin mortar using appropriate colored glass beads.

The glass beads are preferably contained in an amount of 10 to 200 parts by weight, based on 100 parts by weight of resin solid content, more preferably in an amount of 50 to 100 parts by weight. If the content is less than 10 parts by weight, flowing property of a resin mortar decreases, and strength and hardness may decrease after cured. And, if the content is more than 200 parts by weight, strength decreases and they may be missed after cured. It is preferable to use increased amount of glass beads for a floor to which much load is given, and to use decreased amount for a thin resin mortar construction.

The glass powder used in the resin mortar is mixed with a resin to increase a viscosity thereby preventing precipitation or sedimentation of fillers such as glass beads, fills voids between the glass beads to increase impact resistance and tensile force, and inhibits expansion and contraction. And, since the glass powder has hardness of 6 to 7, it enforces surface hardness of resin mortar after cured to increase scratch resistance and give slip-preventing function.

As the glass powder, those of various shapes and sizes can be used. The glass powder is obtained by pulverizing a common glass and its composition is not specifically limited so long as it is compatible with a resin such as A, C, E alkali resistant glass powder compositions, etc. The particle size of the glass powder is preferably 10 μm to 1 mm, and average particle diameter is preferably smaller than those of the glass beads in order to fill voids between the glass beads. If glass powder of less than 10 μm is used, viscosity increases, and if glass powder of more than 1 mm is used, void-filling is not good hence strength may decrease and contraction and expansion may increase.

The glass powder is preferably contained in an amount of 10 to 400 parts by weight, based on 100 parts by weight of resin solid content, and more preferably in an amount of 50 to 100 parts by weight. If the content is less than 10 parts by weight, the viscosity of the resin mortar decrease, and contraction and expansion increase after cured. And, if the content is more than 400 parts by weight, viscosity increases too much, resin content decreases to lower strength, and glass beads may be missed after resin mortar is cured. The glass powder can be contained in a large amount because it does not absorb resin. It is preferable to use decreased amount of glass powder in order to lower viscosity in case constructed under low temperature conditions, to use increased amount for a floor to which much load is applied, and to use decreased amount for a thin resin mortar construction.

The glass fiber used in the present invention exists in a resin to increase tensile force of cured resin mortar and prevent cracks. As the glass fiber, long glass fiber of E glass composition is preferable, and alkali resistance fiber can be used. And, a chopped fiber that is prepared by cutting a glass fiber with a fiber diameter of 10 to 20 μm to uniform stand lengths, or a milled fiber prepared by milling to a average fiber lengths can be used. The chopped fiber is preferably those cut to fiber length of 2 to 12 mm, and the milled fiber is preferably those having average fiber length of 100 to 300 μm. The milled fiber is preferable in terms of tensile force enforcement and dispersion, and the combination of the chopped fiber and milled fiber can be used.

The glass fiber is preferably contained in an amount of 1 to 50 parts by weight, based on 100 parts by weight of resin solid content. If the content is less than 1 part by weight, tensile force of the cured resin mortar decreases, cracks are generated, and contraction and expansion increase. And, if the content is more than 50 parts by weight, mixing and dispersion is difficult.

In order to control construction operability of the resin mortar, a solvent such as benzyl alcohol can be added to control viscosity. The solvent is selected according to a kind of room temperature curable organic liquid phase resin, and it is preferably added in an amount of 1 to 1000 parts by weight based on 100 parts by weight of the resin solid content. The viscosity is suitably 5000 to 10000 cps in case a resin mortar is coated on a floor, and 15000 to 20000 cps in case coated on a wall.

In order to give color to the resin mortar, colored glass bead can be used, or a pigment or color chip can be added. The pigment and the color chip is preferably added in an amount of 0.1 to 20 parts by weight, based on 100 parts by weight of resin solid content in terms of mixing and dispersion, and blending stability, and the pigments is more preferably added in an amount of 0.1 to 5 parts by weight. As the color chip, basic color chips such as white, black or other colors contained in artificial granite, etc., and as the resin used in the color chip, polymethylmethacryalte or polyester is preferable in terms of compatibility with the resin. And, natural granite pattern can be easily obtained by introducing various color chips of various sizes and colors.

The resin mortar of the present invention can be used for floors, wall finishing, waterproof agent, floor surface repairing agent, load repairing agent, etc., and it has superior abrasion resistance and adhesion to constructed body, and thus it can finish a floor surface beautifully even if constructed with a thin thickness of 0.3 to 5 mm on a cement concrete such as a commercial building, a factory floor, a parking lot floor, etc., or on a steel such as ships or automobiles.

The method for constructing a resin mortar on a floor surface will now be explained.

The conventional resin mortar using silica as filler has insufficient silica flowing property, and thus it cannot be stored and used after mixing due to precipitation and tangle. However, in the resin mortar of the present invention, although primary precipitation forms during storage due to self gravity of glass bead fillers, since precipitates move because of superior flowing property if a direction of storage container is changed, the precipitates are not solidified and dispersed again, hence the resin mortar of the present invention can be stored and used for a long period. Therefore, construction can be completed by adding in place resin additives such as a curing agent, curing promoter, etc. to a resin mortar prepared in the factory, and then immediately coating it a floor surface, removing bubbles generated from the resin, and curing it. And, since the resin mortar of the present invention has superior flowing property and thus self-leveling property, coating is completed simply by pouring the resin mortar onto a floor and flattening the surface of the resin mortar using simple equipment such as a rake, etc.

If the resin mortar of the present invention is coated on a wide floor, bubbles form from the resin due to hidden bubbles in concrete and resin properties, which may remain as pockmarks on a resin mortar surface and cause floor contamination and cracks. Therefore, in order to remove such bubbles, glass beads are sprayed together with compressed air before the coated resin mortar is cured. The spraying of the glass beads breaks bubbles to completely remove bubbles. And, the glass beads sprayed onto a surface are deposited into the resin mortar or remain on the surface due to superior compatibility and flowing property, thereby obtaining smooth surface again. The spraying is conducted by spouting the glass beads on the upper part of the coated resin mortar together with air under pressure of 1 to 10 kgf/cm² using a spray equipment connected to a compressor. The amount of the sprayed glass beads are preferably set to 10 to 100 g/m². Therefore, the upper part of the resin mortar contains glass beads in a higher density than the lower part.

Before the resin mortar of the present invention is coated on a floor surface, common primer coating can be conducted on a floor surface according to the conditions of floor surface. The primer coating material is selected according to floor surface and resin material, and for a cement floor surface, epoxy-based, acryl-based, or urethane-based emulsion primer is preferable.

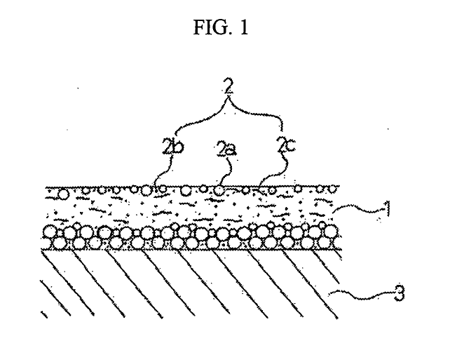

FIG. 1 is a cross-sectional view showing one example wherein the resin mortar of the present invention is constructed on a cement concrete floor surface. In the resin (1), glass beads (2a), glass powder (2b) and glass fiber (2c) are dispersed as fillers (2) to form a cured body. And, as the glass beads (2a), those of various sizes can be used together, and a part of them can be protruded on the surface of the cured body.

If the resin mortar of the present invention is constructed on a floor, it can be firmly adhered to most floors regardless of floor material, and after cured, it can be cleanly maintained as indoor paper-covered floor because of its superior contamination resistance, and it can finish floor for heavy walking because of its superior hardness and strength. And, the construction method of the present invention can obtain a floor that has beautiful surface and does not generate cracks.

The present invention will be explained in detail with reference to the following Examples. However, these are only to illustrate the present invention and the present invention is not limited to them.

### EXAMPLES

### Example 1

(Preparation of Resin Mortar)

1 kg of epoxy liquid phase resin (Kuk-do Chemicals YD-128) and 20 g of benzyl alcohol were mixed, and 1000 g of glass beads with particle diameter distribution of 0.3 to 0.5 mm (Jisan Industry), 1200 g of glass powder with average particle diameter of 200 meshes and specific gravity of 2.54 (Kumyoong Industry Co. Ltd.), 50 g of milled glass fiber with average fiber thickness of 13.5 μm, and average fiber length of 300 μm (Kumyoong Industry Co. Ltd. MF300), and 150 g of green pigment were mixed therewith in a common mixer to prepare a resin mortar.

The prepared resin mortar was green, and its specific gravity was 1.3, 600 gloss was 85%, and flowing property was 50 cm as result of slump test. The resin mortar is put in a steel cam, and stored at room temperature for 12 months, and then it is opened. As results, it was observed that fillers are partially precipitated but they are not solidified, and when shaking the can, the fillers are uniformly dispersed again, and slump test result showed 50 cm.

### Example 2

(Floor Construction of Resin Mortar)

After cleaning common cement concrete floor surface, an epoxy-based emulsion primer (Carboline Korea, KOP coating 340 gold primer) was coated and naturally cured. After one hour, the resin mortar prepared in the Example 1 was mixed with 400 g of amine type curing agent (Kukdo Chemical G-715) and 4 g of amine type cure-promoter (German Huntzman Company, AEP), and then it was poured onto the floor surface and the surface was flattened to a thickness of 5 mm using a rake. Then, the same glass beads as used in the Example 1 were sprayed onto the upper part of the resin mortar in an amount of 10 g/m² under pressure of 2 kgf/cm² using a paint spray connected to a compressor, thereby removing bubbles. The resin mortar was cured at room temperature for 8 hours to obtain a final floor surface wherein resin mortar is constructed to a thickness of 5 mm on a cement concrete.

The surface of the obtained green floor surface was smooth and 60° gloss was 85%.

The other physical properties of the floor surface were described in the following Table 1.

TABLE 1

| | Results |
|---|---|
| In water adhesion strength (kgf/cm²) | 32.6 |
| Wet adhesion strength (kgf/cm²) | 37.8 |
| Dry adhesion strength (kgf/cm²) | 97.5 |
| Tensile force (kgf/cm²) | 200 |
| Bending strength (kgf/cm²) | 510 |
| Compression strength (kgf/cm²) | 860 |
| Hardness (shore-D) | 89 |
| Shear adhesion strength (kgf/cm²) | 87 |

The physical properties were measured according to JIS-K6911.

In addition, the same resin mortar mixture as the above except excluding cure-promoter was coated on a steel plated to a thickness of 30 μm, and was completely cured at 20° C. for 1 day, and then physical properties were measured according to JIS-K6911. The results were described in the following Table 2.

TABLE 2

| | Results |
|---|---|
| drying time (hr, at 25° C.) | Within 8 hrs. |
| Pencil hardness (H) | 1˜2 |
| Cross cut test (RO/100) | 100/100 |
| Bending test (φ 3 mm, 180) | Pass |
| Adhesive test (number/10) | 10/10 |
| Erichsen test (mm) | 5.7 |

In addition, the same mixture as the above except excluding a pigment and a cure-promoter was coated on a release paper to prepare a sample of 10×10×120 mm, and was completely cured at 20° C. for 1 hour, and then chemical resistance was evaluated. The sample was immersed in various chemical solutions as shown in the following Table 3 for 1 month, and then taken out to measure weight decrease.

TABLE 3

| | Weight decrease rate |
|---|---|
| | (wt %) |
| Boiling water | 0.0001 |
| NaOH aqueous solution of 40 wt % concentration | −0.0035 |
| HCl aqueous solution of 36 wt % concentration | 1.065 |
| H₂SO₄ aqueous solution of 50 wt % concentration | 0.188 |
| Acetic acid aqueous solution of 10 wt % | −0.035 |
| concentration | |
| seawater solution | 0.362 |
| Xylene | 0.068 |
| Ethanol | 0.645 |
| K₂Cr₂O₅ of 10 wt % concentration | −0.068 |

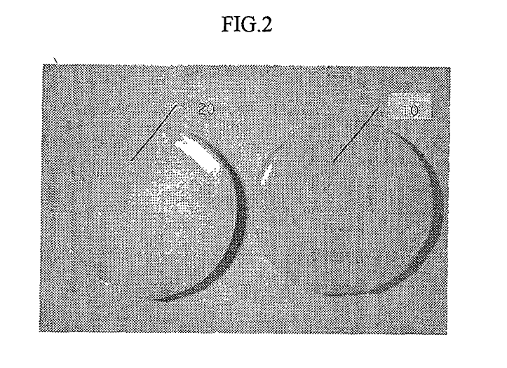

In addition, the same mixture as the above except excluding a pigment and a cure-promoter was coated on a release paper to prepare a spherical sample with a thickness of 5 mm (φ 100), and then was completely cured at 20° C. for 1 day. Then, abrasion resistance was tested by 20,000 times of repetitive frictions using a brush. As results, the coating of the resin mortar maintained without break and loss, and weight decrease was 0.015 wt %. The appearance of the sample was shown in FIG. 2. Reference numeral 10 indicates a sample before conducting a test, and reference numeral 20 a sample after test.

### Example 3

The resin mortar was prepared with the same composition as in Example 1, except that 30 g of combination color chip of white, black, blue and red was mixed instead of a pigment, and the prepared resin mortar was coated on a cement concrete floor by the same method as in Example 2 and cured to obtain a floor with natural granite pattern. The physical properties of the floor were identical with those in Example 2.

The resin mortar of the present invention uses glass beads having superior flowing property and high hardness as a filler and thus has superior self-leveling property when constructed, and superior abrasion resistance, scratch resistance, crack resistance and durability after constructed, and it can be easily constructed and easily control curing time. The construction method using the composition can obtain a floor that has beautiful surface and does not generate cracks.

What is claimed is:

1. A resin mortar composition for construction comprising:
on the basis of solid content,
a) 10 parts by weight of a room temperature curable organic liquid phase resin;
b) 10 to 200 parts by weight of glass beads;
c) 10 to 400 parts by weight of glass powder; and
d) 0.1 to 50 parts by weight of glass fiber.

2. The resin mortar composition according to claim 1 further comprising e) 0.1 to 20 parts by weight of a pigment or a color chip.

3. The resin mortar composition according to claim 1 further comprising f) 1 to 100 parts by weight of a solvent.

4. The resin mortar composition according to claim 1, wherein the a) room temperature curable organic liquid phase resin is selected from the group consisting of an epoxy-based resin, an acryl-based resin, an urethane-based resin, an alkyd-based resin, a polyester-based resin and a polyvinylchloride-based resin.

5. The resin mortar composition according to claim 1, wherein the b) glass beads have particle sizes of 200 meshes to 3 mm.

6. The resin mortar composition according to claim 1, wherein the c) glass powder has particle size of 10 μm to 1 mm.

7. The resin mortar composition according to claim 1, wherein the d) glass fiber is chopped fiber that is prepared by cutting glass long fiber of E-glass composition to a length of 2 to 12 mm, or a milled fiber that is prepared by milling to a length of 100 to 300 μm.

8. A method for constructing a resin mortar on a floor, which comprises the steps of:
a) coating a resin mortar comprising:
on the basis of solid content,
i) 10 parts by weight of a room temperature curable organic liquid phase resin;
ii) 10 to 200 parts by weight of glass beads;
iii) 10 to 400 parts by weight of glass powder; and
iv) 0.1 to 50 parts by weight of glass fiber
on a floor;
b) spraying glass beads on the floor that is coated with the resin mortar to remove bubbles produced in the coated resin mortar; and
c) curing the resin mortar floor from which bubbles are removed.

9. The method according to claim 8, wherein a primer coating is conducted on a floor before the step a).

10. The method according to claim 8, wherein the resin mortar of the step a) further comprises v) 0.1 to 20 parts by weight of a pigment or a color chip.

11. The method according to claim 8, wherein in the step b), glass beads are sprayed in an amount of 1 to 100 g/m² together with air having pressure of 1 to 10 kgf/cm².
